(19) United States
(12) Patent Application Publication
(10) Pub. No.: US 2025/0325784 A1
(43) Pub. Date: Oct. 23, 2025

(54) CATHETER, CATHETER SYSTEM, AND TREATMENT METHOD

(71) Applicant: TERUMO KABUSHIKI KAISHA, Tokyo (JP)

(72) Inventors: Takashi ITO, Shizuoka-shi (JP); Mitsuteru YASUNAGA, Fujinomiya-shi (JP); Toshiki ASAWA, Fujinomiya-shi (JP)

(73) Assignee: TERUMO KABUSHIKI KAISHA, Tokyo (JP)

(21) Appl. No.: 19/257,648

(22) Filed: Jul. 2, 2025

Related U.S. Application Data
(63) Continuation of application No. PCT/JP2023/042135, filed on Nov. 24, 2023.

(30) Foreign Application Priority Data
Jan. 30, 2023 (JP) 2023-011579

(51) Int. Cl. A61M 25/00; A61B 17/00; A61B 17/22

(52) U.S. Cl. CPC A61M 25/0045; A61B 17/00234; A61B 17/22; A61B 2017/00238; A61B 2017/00305; A61B 2017/0084; A61B 2017/22038; A61M 2025/0006; A61M 2025/0046; A61M 2205/0222; A61M 2205/0238; A61M 2210/12

(57) ABSTRACT

A catheter for supporting a lesion passage catheter capable of improving ease of passage of the lesion passage catheter through a lesion of a vein, a catheter system, and a treatment method. The support catheter includes an elongated third tubular body used for passage through a lesion of a vein, in which an inner diameter of the third tubular body is not less than 1.4 mm and an outer diameter of the third tubular body is not more than 2.8 mm.

### CROSS-REFERENCES TO RELATED APPLICATIONS

This application is a continuation of International Application No. PCT/JP2023/042135 filed on Nov. 24, 2023, which claims priority to Japanese Patent Application No. 2023-011579 filed on Jan. 30, 2023, the entire content of both of which is incorporated herein by reference.

### TECHNOLOGICAL FIELD

The present disclosure generally relates to a catheter, a catheter system, and a treatment method.

### BACKGROUND DISCUSSION

In the treatment of a blood vessel, when a guide wire passes through a lesion, in a state where the guide wire is inserted into and supported by a device (lesion passage catheter) into which the guide wire is insertable, a procedure of causing the lesion passage catheter to pass through the lesion together with the guide wire is performed (See, for example, Japanese Patent Application Publication No. 2005-533554 A).

Meanwhile, a lesion passage catheter for an artery is diverted in treatment of deep vein thrombosis (DVT). The target blood vessel for deep vein thrombosis is a vein on the peripheral side of the inferior vena cava, and can be, for example, an iliac vein, a femoral vein, a popliteal vein, or the like. The blood vessel diameter of a vein is generally greater than the blood vessel diameter of an artery. For example, the inner diameter of an artery that accesses the catheter is about 3 Fr to 8 Fr, whereas the inner diameter of a vein that accesses the catheter is about 6 Fr to 24 Fr.

Since a lesion passage catheter for accessing a lesion of a vein, the lesion passage catheter being adapted to the blood vessel diameter and the lesion of the vein is not known, when a thin lesion passage catheter for an artery is used in a vein, the lesion passage catheter is likely to bend in the thick vein, and ease of passage through a lesion may be lowered. In order to pass through an organized thrombus, the lesion passage catheter is preferably relatively thin. Therefore, in order to use a thin lesion passage catheter in a thick vein, a guiding catheter for guiding the lesion passage catheter is required. However, when the outer diameter of the lesion passage catheter is too small in comparison with the inner diameter of the parent catheter adapted to the blood vessel diameter of the vein, the lesion passage catheter bends inside the parent catheter, and ease of passage of the lesion passage catheter through the lesion is lowered.

### SUMMARY

A catheter is disclosed, which supports a lesion passage catheter capable of improving ease of passage of the lesion passage catheter through a lesion of a vein, a catheter system, and a treatment method.

(1) A catheter according to the present disclosure is a catheter including an elongated tubular body used for passage through a lesion of a vein, in which an inner diameter of the elongated tubular body is not less than 1.4 mm and an outer diameter of the elongated tubular body is not more than 2.8 mm.

The catheter described in (1) can satisfactorily support a lesion passage catheter having an upper limit of an outer diameter of about 1.4 mm, the outer diameter being considerably smaller than an inner diameter of a guiding catheter inside the parent catheter having a lower limit of the inner diameter of about 2.8 mm in conformity with a vein. Therefore, the catheter can improve ease of passage of the lesion passage catheter through a lesion of a vein.

(2) In the catheter according to (1), the outer diameter of the elongated tubular body may be not more than 2.0 mm. As a result, the catheter can satisfactorily support the lesion passage catheter inside the parent catheter having the lower limit of the inner diameter of about 2.0 mm in conformity with a vein.

(3) In the catheter according to (1) or (2), an outer peripheral surface of at least a distal end portion of the elongated tubular body may be coated with a lubricious coat. As a result, the outer peripheral surface of the distal end portion of the tubular body has low friction. A thrombus formed in a vein tends to be formed longer in a length direction of the blood vessel than a thrombus formed in an artery. Therefore, the fact that the outer peripheral surface of the tubular body becomes low friction by covering the outer peripheral surface of the tubular body with the lubricious coat is very effective when causing the tubular body to pass through a long thrombus of a vein.

(4) In the catheter according to any one of (1) to (3), an effective length of the elongated tubular body may be less than 1000 mm. As a result, the catheter is effective for treating a deep vein in which the length from the access position to the blood vessel to the lesion position is likely to be less than 1000 mm. The longer the effective length, the lower the operability of the catheter, so that the effective length is desirably short as long as no problem occurs.

(5) A catheter system for achieving the above object is a catheter system used for passage through a lesion of a vein, the catheter system including: a catheter; a lesion passage catheter that is insertable into the catheter; a guiding catheter into which the catheter is insertable; an inner catheter that is insertable into the lesion passage catheter; and an outer catheter into which the guiding catheter is insertable, in which tubular bodies of the inner catheter, the lesion passage catheter, the catheter, the guiding catheter, and the outer catheter are coaxially overlapped in this order from inside to outside. As a result, the catheter system can improve ease of passage of the lesion passage catheter and a guide wire through a lesion of a vein by conforming to the vein and pushing the catheter system coaxially with the vein.

(6) A catheter system for achieving the above object is a catheter system used for passage through a lesion of a vein, the catheter system including: a catheter including an elongated tubular body having an inner diameter of not less than 1.4 mm and an outer diameter of not more than 2.8 mm; a lesion passage catheter that is insertable into the elongated tubular body of the catheter; and a guiding catheter into which the catheter is insertable. As a result, the catheter system can satisfactorily support the lesion passage catheter having an upper limit of an outer diameter of about 1.4 mm, the outer diameter being considerably smaller than an inner diameter of a guiding catheter inside the parent catheter having a lower limit of the inner diameter of about 2.8 mm in conformity with a vein. Therefore, the catheter system can improve ease of passage of the lesion passage catheter through a lesion of a vein.

(7) The catheter system according to (6) may further include: an inner catheter that is insertable into the lesion passage catheter; and an outer catheter into which the guiding catheter is insertable. As a result, the catheter system can further improve ease of passage of the lesion passage catheter through a lesion of a vein.

(8) A treatment method for causing at least part of a catheter system to pass through a lesion of a vein together with a guide wire, the treatment method including: allowing passage through a lesion of a vein by the catheter system in which an elongated tubular body of a catheter is inserted into a guiding catheter, the catheter including the elongated tubular body having an inner diameter of not less than 1.4 mm and an outer diameter of not more than 2.8 mm and a lesion passage catheter is inserted into the elongated tubular body. As a result, in the treatment method, the lesion passage catheter having an upper limit of an outer diameter of about 1.4 mm, the outer diameter being considerably smaller than an inner diameter of the guiding catheter can be satisfactorily supported inside the guiding catheter having a lower limit of the inner diameter of about 2.8 mm in conformity with a vein. Therefore, the treatment method can improve ease of passage of the catheter system through a lesion of a vein.

(9) In the treatment method according to (8), passage through a lesion of a vein may be allowed by the catheter system in which the guiding catheter is inserted into an outer catheter, and an inner catheter is inserted into the lesion passage catheter. Therefore, the treatment method can further improve ease of passage of the lesion passage catheter through a lesion of a vein.

### BRIEF DESCRIPTION OF THE DRAWINGS

FIG. 1 is a plan view illustrating a state in which a catheter system according to an embodiment is assembled.

FIG. 2 is a plan view illustrating a state in which the catheter system according to the embodiment is separated.

FIG. 3 is a cross-sectional view taken along line A-A in FIG. 1.

### DETAILED DESCRIPTION

An embodiment of the present disclosure will be described below with reference to the drawings. Note that, dimensional ratios in the drawings are sometimes exaggerated and different from actual ratios for convenience of description. In the following description, a side on which a catheter is operated is referred to as a “proximal end side”, and a side to be inserted into a living body is referred to as a “distal end side”.

A catheter system 10 according to an embodiment of the present disclosure is a device used for passage of a guide wire through a lesion in the treatment of deep vein thrombosis (DVT). The catheter system 10 is inserted from a vein in a knee or the like of the living body and pushed into a blood vessel to be treated. The blood vessel to be treated is a vein (for example, an iliac vein, a femoral vein, a popliteal vein, or the like) on the peripheral side of the inferior vena cava.

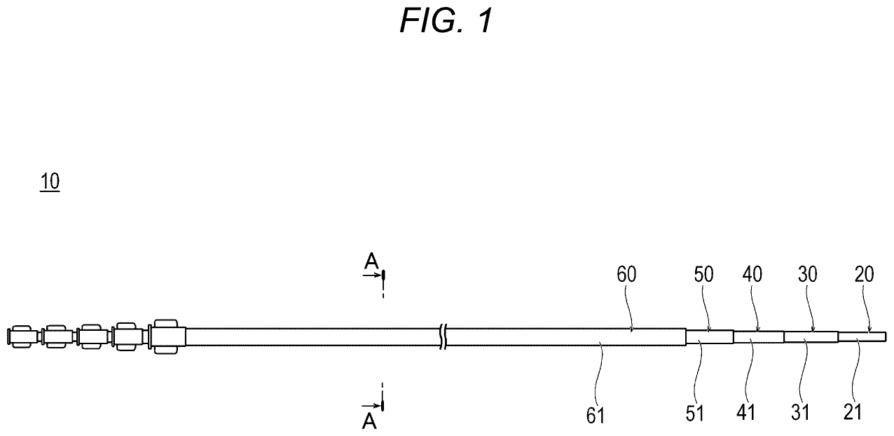

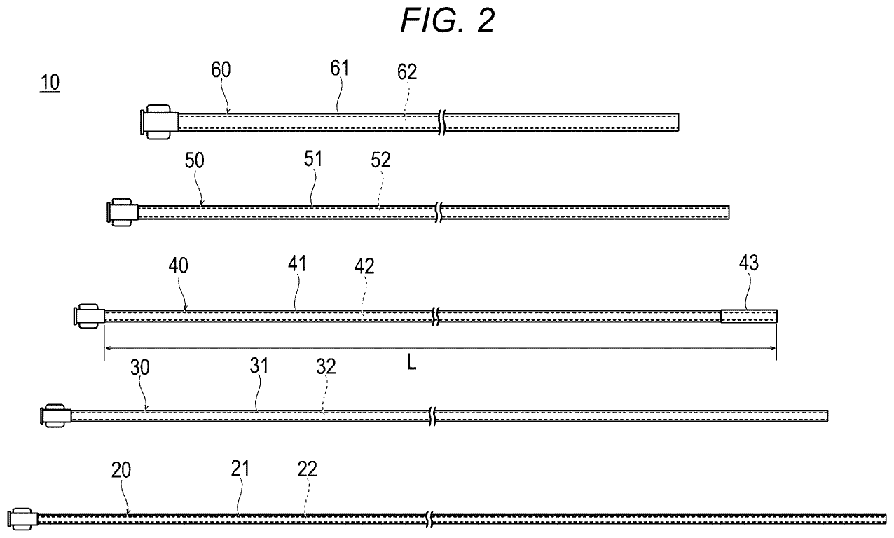

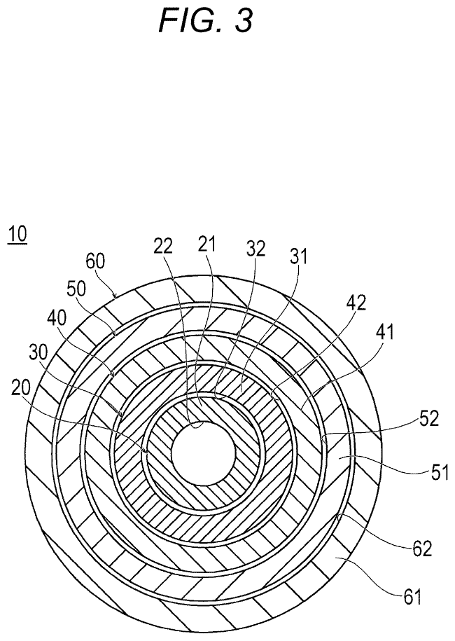

As illustrated in FIGS. 1 to 3, the catheter system 10 includes a lesion passage catheter 30, an inner catheter 20 inserted into the lesion passage catheter 30, a support catheter 40 (catheter) that supports the lesion passage catheter 30, a guiding catheter 50 that guides the support catheter 40, and an outer catheter 60 that guides the guiding catheter 50. In the catheter system 10, tubular bodies of the inner catheter 20, the lesion passage catheter 30, the support catheter 40, the guiding catheter 50, and the outer catheter 60 are coaxially overlapped in this order from inside to outside. Note that the catheter system 10 may not include the outer catheter 60 and/or the inner catheter 20.

The lesion passage catheter 30 has a second tubular body 31 in which a second lumen 32 into which a guide wire caused to pass through a lesion of a vein is insertable is formed. The lesion passage catheter 30 covers and supports the guide wire inserted into the second lumen 32 and is used for passage through a lesion together with the guide wire. Instead of the guide wire, the inner catheter 20 is insertable into the second lumen 32. In this case, the lesion passage catheter 30 covers and supports the inner catheter 20 in order to cause the inner catheter 20 and a thinner guide wire insertable into the inner catheter 20 to pass through a lesion and is used for passage through the lesion together with the inner catheter 20 and the guide wire.

The inner catheter 20 has a first tubular body 21 in which a first lumen 22 into which the guide wire caused to pass through a lesion of a vein is insertable is formed. The inner catheter 20 covers and supports the guide wire inserted into the first lumen 22 and is used for passage through a lesion together with the guide wire. The guide wire insertable into the inner catheter 20 is thinner than the guide wire insertable into the lesion passage catheter 30. The inner catheter 20 is inserted into the second lumen 32 of the lesion passage catheter 30 and used in order to support the thinner guide wire in a case where the thick guide wire inserted into the lesion passage catheter 30 cannot pass through a lesion, for example. Note that the inner catheter 20 may not be used.

The support catheter 40 (catheter) has a third tubular body 41 (tubular body) in which a third lumen 42 into which the lesion passage catheter 30 is insertable is formed. The support catheter 40 supports the lesion passage catheter 30 inserted into the third lumen 42, the inner catheter 20, and the guide wire inserted into the inner catheter 20, and is used for passage through a lesion together with the lesion passage catheter 30, the inner catheter 20, and the guide wire. Note that in a case where the inner catheter 20 is not inserted into the lesion passage catheter 30, the support catheter 40 supports the lesion passage catheter 30 inserted into the third lumen 42 and the guide wire inserted into the lesion passage catheter 30, and is used for passage through a lesion together with the lesion passage catheter 30 and the guide wire. Note that the support catheter 40 may or may not completely pass through a lesion when the lesion passage catheter 30 and the like supported by the support catheter 40 pass through the lesion.

The outer peripheral surface of at least the distal end portion of the third tubular body 41 may be covered with a lubricious coat 43. The lubricious coat 43 can be, for example, a hydrophilic polymer, and examples of the lubricious coating 43 can include a cellulose-based polymer substance, a polyethylene oxide-based polymer substance, a maleic anhydride-based polymer substance (for example, a maleic anhydride copolymer such as a methyl vinyl ether-maleic anhydride copolymer), an acrylamide-based polymer substance (for example, polyacrylamide, a block copolymer of glycidyl methacrylate-dimethylacrylamide), water-soluble nylon, polyvinyl alcohol, polyvinyl pyrrolidone, and derivatives of the lubricious coatings listed above. The hydrophilic polymer forms a strong water fixing layer on the surface of outer peripheral surface of at least the distal end portion of the third tubular body 41, exhibits high affinity to blood in a blood vessel and a wall surface of the blood vessel, and exhibits low friction. Alternatively, the lubricious coat 43 may be a low friction material or the like such as a fluorine-based resin such as polytetrafluoroethylene (PTFE), high density polyethylene (HDPE), or the like. Meanwhile, a thrombus formed in a vein tends to be formed longer in a length direction of the blood vessel than a thrombus formed in an artery. Therefore, the fact that the outer peripheral surface of the third tubular body 41 has low friction is very effective when the third tubular body 41 passes through a vein thrombus.

The effective length L of the support catheter 40 is not particularly limited, but is preferably less than 1000 mm. The effective length L of the support catheter 40 is a length of a portion insertable into a blood vessel or a tubular device. In the present embodiment, the effective length L of the support catheter 40 is a length from the most distal end of a hub or a strain relief at the proximal end portion to the most distal end of the third tubular body 41.

The guiding catheter 50 has a fourth tubular body 51 in which a fourth lumen 52 into which the support catheter 40 is insertable is formed. The guiding catheter 50 is used to secure a passage in a blood vessel. Alternatively, the guiding catheter 50 supports the support catheter 40 inserted into the fourth lumen 52 and the other catheters and the guide wire inside the support catheter 40, and is used to assist at least part of them to pass through a lesion. The guiding catheter 50 can be, for example, an introducer sheath or a guiding sheath.

The outer catheter 60 has a fifth tubular body 61 in which a fifth lumen 62 into which the guiding catheter 50 is insertable is formed. The outer catheter 60 is used to secure a passage in a blood vessel. Alternatively, the outer catheter 60 supports the guiding catheter 50 inserted into the fifth lumen 62 and the other catheters and the guide wire inside the guiding catheter 50, and can be used to assist at least part of the inner catheter 20, the lesion passage catheter 30, and the support catheter 40 to pass through a lesion. The outer catheter 60 can be, for example, an introducer sheath or a guiding sheath. Note that the outer catheter 60 may not be used.

Examples of the inner diameter and the outer diameter of each catheter are indicated in Table 1.

TABLE 1

| | | Lesion | | | |
|---|---|---|---|---|---|
| | | passage | Support | Guiding | |
| | Inner catheter | catheter | catheter | catheter | Outer catheter |
| Inner diameter | Inner diameter: | Inner diameter: | Inner diameter: | Inner diameter: | Inner diameter: |
| and outer | 0.36 to 0.60 | 0.86 to 1.2 | 1.4 to 1.6 | 1.6 to 2.5 | 2.8 to 3.0 |
| diameter (mm) | Outer diameter | Outer diameter | Outer diameter | Outer diameter | Outer diameter |
| | 0.70 to 0.90 | 1.2 to 1.4 | 1.6 to 2.0 | 1.8 to 2.8 | 3.0 to 3.6 |
| | | | | (guiding sheath | (guiding sheath |
| | | | | of 6 Fr) | of 8 Fr) |
| | | | Inner diameter: | Inner diameter: | — |
| | | | 1.4 to 2.5 | 2.8 to 3.0 | |
| | | | Outer diameter | Outer diameter | |
| | | | 2.0 to 2.8 | 3.0 to 3.6 | |
| | | | | (guiding sheath | |
| | | | | of 8 Fr) | |
| Role of device | Specialized in | Specialized in | Passage | Support of | Support of |
| | passage | passage | through lesion | system | system |
| | through lesion | through lesion | and | | |
| | | | Support of | | |
| | | | inner catheter | | |
| | | | and lesion | | |
| | | | passage | | |
| | | | catheter | | |

The inner diameter of the first tubular body 21 of the inner catheter 20 is not particularly limited, but is preferably 0.36 mm to 0.60 mm. The outer diameter of the first tubular body 21 is not particularly limited, but is preferably 0.70 mm to 0.90 mm.

The inner diameter of the second tubular body 31 of the lesion passage catheter 30 is not particularly limited, but is preferably 0.86 mm to 1.2 mm. The outer diameter of the second tubular body 31 is not particularly limited, but is preferably 1.2 mm to 1.4 mm.

The inner diameter and the outer diameter of the third tubular body 41 of the support catheter 40 are not particularly limited, but are appropriately set according to the dimensions of the guiding catheter 50 into which the support catheter 40 is to be inserted. For example, in a case where the guiding catheter 50 is a guiding sheath of 6 Fr, the inner diameter of the third tubular body 41 is preferably 1.4 mm to 1.6 mm, and the outer diameter of the third tubular body 41 is preferably 1.6 mm to 2.0 mm.

In a case where the guiding catheter 50 is a guiding sheath of 8 Fr, the inner diameter of the third tubular body 41 is preferably 1.4 mm to 2.5 mm, and the outer diameter of the third tubular body 41 is preferably 2.0 mm to 2.8 mm. That is, comprehensively, the inner diameter of the third tubular body 41 of the support catheter 40 is preferably 1.4 mm to 2.5 mm, and the outer diameter of the third tubular body 41 is preferably 1.6 mm to 2.8 mm.

The inner diameter and the outer diameter of the fourth tubular body 51 of the guiding catheter 50 are not particularly limited, but in a case where the guiding catheter 50 is a guiding sheath of 6 Fr, the inner diameter of the fourth tubular body 51 is preferably 1.6 mm to 2.5 mm, and the outer diameter of the fourth tubular body 51 is preferably 1.8 mm to 2.8 mm. In a case where the guiding catheter 50 is a guiding sheath of 8 Fr, the inner diameter of the fourth tubular body 51 is preferably 2.8 mm to 3.0 mm, and the outer diameter of the fourth tubular body 51 is preferably 3.0 mm to 3.6 mm. That is, comprehensively, the inner diameter of the fourth tubular body 51 of the guiding catheter 50 is preferably 1.6 mm to 3.0 mm, and the outer diameter of the fourth tubular body 51 is preferably 1.8 mm to 3.6 mm.

The inner diameter and the outer diameter of the fifth tubular body 61 of the outer catheter 60 are not particularly limited, but in a case where the outer catheter 60 is a guiding sheath of 8 Fr, the inner diameter of the fifth tubular body 61 is preferably 2.8 mm to 3.0 mm, and the outer diameter of the fifth tubular body 61 is preferably 3.0 mm to 3.6 mm.

The outer diameter of the inner catheter 20 is less than the inner diameter of the lesion passage catheter 30, the outer diameter of the lesion passage catheter 30 is less than the inner diameter of the support catheter 40, the outer diameter of the support catheter 40 is less than the inner diameter of the guiding catheter 50, and the outer diameter of the guiding catheter 50 is less than the inner diameter of the outer catheter 60.

The combination patterns of the respective catheters are indicated in Table 2.

TABLE 2

| | | Lesion | | | | | |
|---|---|---|---|---|---|---|---|
| | Inner | passage | Support | Guiding | Outer | | |
| Pattern | catheter | catheter | catheter | catheter | catheter | Sheath | Remarks |
| A-1 | Optional | Indispensable | Optional | — | — | 6 Fr | After passage of |
| | | | | | | | guide wire |
| | | | | | | | through lesion, |
| | | | | | | | sheath is upsized |
| | | | | | | | for device for |
| | | | | | | | IVUS or |
| | | | | | | | Thrombectomy |
| | | | | | | | device |
| A-2 | Optional | Indispensable | Optional | Indispensable | — | — | Approach without |
| | | | | | | | sheath |
| B-1 | Optional | Indispensable | Optional | Optional | — | 8 Fr | After passage of |
| | | | | | | | guide wire |
| | | | | | | | through lesion, |
| | | | | | | | sheath is upsized |
| | | | | | | | for device for |
| | | | | | | | IVUS or |
| | | | | | | | Thrombectomy |
| | | | | | | | device |
| B-2 | Optional | Indispensable | Optional | Optional | Indispensable | — | Approach without |
| | | | | | | | sheath |
| C | Optional | Indispensable | Optional | Optional | Optional | Not less | — |
| | | | | | | than 12 | |
| | | | | | | Fr | |
| Role of | Support | Support | Support | Secure | Secure | Secure | — |
| device | passage of | passage of | passage of | blood | blood | blood | |
| | guide wire | guide wire | guide wire | vessel | vessel | vessel | |
| | through | through | through | (A-2) | (B-2) | A-1 | |
| | lesion | lesion | lesion | or | or | B-1 | |
| | | or | and | Support | Support | C | |
| | | Support | Support | support | guiding | | |
| | | inner | inner | catheter | catheter | | |
| | | catheter | catheter | and rest | and rest | | |
| | | | and lesion | | | | |
| | | | passage | | | | |
| | | | catheter | | | | |

In pattern A-1, a sheath of 6 Fr is used to secure a blood vessel, and the guiding catheter 50 and the outer catheter 60 are not used. The lesion passage catheter 30 is indispensably used, and the inner catheter 20 and the support catheter 40 are optionally used. After the guide wire has passed through a lesion, the sheath is upsized to insert a device for intravascular ultrasound (IVUS) or a device for thrombectomy. In a case where the inner catheter 20, which is optionally is used, the lesion passage catheter 30 plays a role of supporting the inner catheter 20 and the rest (the inner catheter 20 and the guide wire inside the inner catheter 20). In a case where the inner catheter 20 is not used, the lesion passage catheter 30 plays a role of supporting the guide wire. The support catheter 40 plays a role of supporting the lesion passage catheter 30 and the rest (the lesion passage catheter 30 and all of the catheters, i.e., inner catheter 20 and the guide wire inside the lesion passage catheter 30).

Pattern A-2 differs from Pattern A-1 only in that a sheath is not used and the guiding catheter 50 is used. That is, pattern A-2 is performed by an approach without a sheath. In pattern A-2, the lesion passage catheter 30 and the guiding catheter 50 are indispensably used, and the inner catheter 20 and the support catheter 40 are optionally used. The guiding catheter 50 is used to secure a blood vessel. The guiding catheter 50 may play a role of supporting the support catheter 40 and the rest (the support catheter 40 and all of the catheters 20, 30 and the guide wire inside the support catheter 40).

In pattern B-1, a sheath of 8 Fr is used to secure a blood vessel and the outer catheter 60 is not used. The lesion passage catheter 30 is indispensably used, and the inner catheter 20, the support catheter 40, and the guiding catheter 50 are optionally used. After the guide wire has passed through a lesion, the sheath is upsized to insert a device for intravascular ultrasound (IVUS) or a device for thrombectomy. In a case where the inner catheter 20, which is optionally used, the lesion passage catheter 30 plays a role of supporting the inner catheter 20 and the support catheter 40 and the guiding catheter 50. In a case where the inner catheter 20 is not used, the lesion passage catheter 30 plays a role of supporting the guide wire. The support catheter 40 plays a role of supporting the lesion passage catheter 30 and the support catheter 40 and the guiding catheter 50. The guiding catheter 50 plays a role of supporting the support catheter 40 and the inner catheter 20 and the lesion passage catheter 30.

Pattern B-2 differs from Pattern B-1 only in that a sheath is not used and the outer catheter 60 is used. That is, pattern B-2 is performed by an approach without a sheath. In pattern B-2, the lesion passage catheter 30 and the outer catheter 60 are indispensably used, and the inner catheter 20, the support catheter 40, and the guiding catheter 50 are optionally used. The outer catheter 60 is used to secure a blood vessel. The outer catheter 60 may play a role of supporting the guiding catheter 50 and the rest (the guiding catheter 50 and all of the catheters 20, 30, 40, and the guide wire inside the guiding catheter 50).

In pattern C, a sheath of not less than 12 Fr is used to secure a blood vessel. The lesion passage catheter 30 is indispensably used, and the inner catheter 20, the support catheter 40, the guiding catheter 50, and the outer catheter 60 are optionally used. In a case where the inner catheter 20, which is optional is used, the lesion passage catheter 30 plays a role of supporting the inner catheter 20. In a case where the inner catheter 20 is not used, the lesion passage catheter 30 plays a role of supporting the guide wire. The support catheter 40 plays a role of supporting the lesion passage catheter 30 and the rest. The guiding catheter 50 plays a role of supporting the support catheter 40 and the rest. The outer catheter 60 plays a role of supporting the guiding catheter 50 and the rest (the support catheter 40 and all of the catheters 20, 30 and the guide wire inside the support catheter 40).

As illustrated in FIG. 1, the catheter system 10 is used to cause the guide wire to pass through a vein lesion (for example, an organizing venous embolus) in a state where the inner catheter 20, the lesion passage catheter 30, the support catheter 40, the guiding catheter 50, and the outer catheter 60 are combined. Note that in the catheter system 10, not all of the catheters 20, 30, 40, 50, 60 may necessarily be combined. For example, the catheter system 10 may not include the inner catheter 20 and/or the outer catheter 60.

As described above, the support catheter 40 (catheter) according to the present embodiment is the support catheter 40 including the long third tubular body 41 (tubular body) used for passage through a lesion of a vein, in which the inner diameter of the third tubular body 41 is not less than 1.4 mm and the outer diameter of the third tubular body 41 is not more than 2.8 mm. As a result, the support catheter 40 can satisfactorily support the lesion passage catheter 30 having an upper limit of the outer diameter of about 1.4 mm, the outer diameter being considerably smaller than the inner diameter of the guiding catheter 50 inside the guiding catheter 50 having a lower limit of the inner diameter of about 2.8 mm in conformity with a vein. Therefore, the support catheter 40 can improve ease of passage of the lesion passage catheter 30 through a lesion of a vein.

The outer diameter of the third tubular body 41 may be not more than 2.0 mm. As a result, the support catheter 40 can satisfactorily support the lesion passage catheter 30 inside the guiding catheter 50 having the lower limit of the inner diameter of about 2.0 mm in conformity with a vein.

The outer peripheral surface of at least the distal end portion of the third tubular body 41 may be covered with the lubricious coat 43. As a result, the outer peripheral surface of the distal end portion of the third tubular body 41 has low friction. A thrombus formed in a vein tends to be formed longer in a length direction of the blood vessel than a thrombus formed in an artery. Therefore, the fact that the outer peripheral surface of the third tubular body 41 has low friction by covering the outer peripheral surface of the third tubular body 41 with the lubricious coat 43 is very effective when causing the third tubular body 41 to pass through a long thrombus of a vein.

The effective length of the third tubular body 41 is less than 1000 mm. As a result, the support catheter 40 is effective for treating a deep vein in which the length from the access position to the blood vessel to the lesion position is likely to be less than 1000 mm. The longer the effective length, the lower the operability of the support catheter 40, so that the effective length is desirably short as long as no problem occurs.

The catheter system 10 is the catheter system 10 used for passage through a lesion of a vein, the catheter system 10 including: the support catheter 40 (catheter); the lesion passage catheter 30 that is insertable into the support catheter 40; the guiding catheter 50 into which the support catheter 40 is insertable; the inner catheter 20 that is insertable into the lesion passage catheter 30; and the outer catheter 60 into which the guiding catheter 50 is insertable, in which the tubular bodies (the first tubular body 21, the second tubular body 31, the third tubular body 41, the fourth tubular body 51, and the fifth tubular body 61) of the inner catheter 20, the lesion passage catheter 30, the support catheter 40, the guiding catheter 50, and the outer catheter 60 are coaxially overlapped in this order from inside to outside. As a result, the catheter system 10 can improve ease of passage of the lesion passage catheter 30 and the guide wire through a lesion of a vein by conforming to the vein and pushing the catheter system 10 coaxially with the vein.

The catheter system 10 is the catheter system 10 used for passage through a lesion of a vein, the catheter system 10 including: the support catheter 40 (catheter) including the third tubular body 41 (tubular body) having the inner diameter of not less than 1.4 mm and the outer diameter of not more than 2.8 mm; the lesion passage catheter 30 that is insertable into the third tubular body 41 of the support catheter 40; and the guiding catheter 50 into which the support catheter 40 is insertable. As a result, the catheter system 10 can satisfactorily support the lesion passage catheter 30 having an upper limit of the outer diameter of about 1.4 mm, the outer diameter being considerably smaller than the inner diameter of the guiding catheter 50 inside the guiding catheter 50 having a lower limit of the inner diameter of about 2.8 mm in conformity with a vein. Therefore, the catheter system 10 can improve ease of passage of the lesion passage catheter 30 through a lesion of a vein.

The catheter system 10 further includes the inner catheter 20 that is insertable into the lesion passage catheter 30 and the outer catheter 60 into which the guiding catheter 50 is insertable. As a result, the catheter system 10 can further improve ease of passage of the lesion passage catheter 30 through a lesion of a vein.

A treatment method in the present embodiment is a treatment method for causing at least part of the catheter system 10 to pass through a lesion of a vein together with the guide wire, the treatment method including: allowing passage through a lesion of a vein by the catheter system 10 in which the long third tubular body 41 (tubular body) of the support catheter 40 (catheter) is inserted into the guiding catheter 50, the support catheter 40 including the third tubular body 41 having the inner diameter of not less than 1.4 mm and the outer diameter of not more than 2.8 mm, and the lesion passage catheter 30 is inserted into the third tubular body 41. As a result, in the treatment method, the lesion passage catheter 30 having an upper limit of the outer diameter of about 1.4 mm, the outer diameter being considerably smaller than the inner diameter of the guiding catheter 50 can be satisfactorily supported inside the guiding catheter 50 having a lower limit of the inner diameter of about 2.8 mm in conformity with a vein. Therefore, the treatment method can improve ease of passage of the lesion passage catheter 30 through a lesion of a vein.

In the treatment method described above, passage through a lesion of a vein may be allowed by the catheter system 10 in which the guiding catheter 50 is inserted into the outer catheter 60, and the inner catheter 20 is inserted into the lesion passage catheter 30. Therefore, the treatment method can further improve ease of passage of the lesion passage catheter 30 through a lesion of a vein.

The present disclosure is not limited to the embodiment described above, and those skilled in the art can make various modifications within the technical idea of the present disclosure. For example, a hub connected to the proximal end portion of the tubular body of each catheter may or may not be provided with a valve body or a port. In addition, a strain relief may be disposed so as to cover a space between the tubular body and the hub of each catheter.

The detailed description above describes embodiments of a catheter, a catheter system, and a treatment method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter comprising:
an elongated tubular body configured for passage through a lesion of a vein; and
wherein an inner diameter of the elongated tubular body is not less than 1.4 mm and an outer diameter of the elongated tubular body is not more than 2.8 mm.

2. The catheter according to claim 1, wherein the outer diameter of the elongated tubular body is not more than 2.0 mm.

3. The catheter according to claim 1, wherein an outer peripheral surface of at least a distal end portion of the elongated tubular body is coated with a lubricious coat.

4. The catheter according to claim 2, wherein an outer peripheral surface of at least a distal end portion of the elongated tubular body is coated with a lubricious coat.

5. The catheter according to claim 1, wherein an effective length of the elongated tubular body is less than 1000 mm.

6. The catheter according to claim 2, wherein an effective length of the elongated tubular body is less than 1000 mm.

7. A catheter system for passage through a lesion of a vein, the catheter system comprising:
a catheter;
a lesion passage catheter that is insertable into the catheter; and
a guiding catheter into which the catheter is insertable.

8. The catheter system according to claim 7, further comprising:
an inner catheter that is insertable into the lesion passage catheter.

9. The catheter system according to claim 8, further comprising:
an outer catheter into which the guiding catheter is insertable.

10. The catheter system according to claim 9, wherein
tubular bodies of the inner catheter, the lesion passage catheter, the catheter, the guiding catheter, and the outer catheter are coaxially overlapped in this order from inside to outside.

11. The catheter system according to claim 7, wherein the catheter includes an elongated tubular body having an inner diameter of not less than 1.4 mm and an outer diameter of not more than 2.8 mm.

12. The catheter system according to claim 11, wherein the lesion passage catheter is insertable into the elongated tubular body of the catheter.

13. The catheter system according to claim 11, wherein the outer diameter of the elongated tubular body of the catheter is not more than 2.0 mm.

14. The catheter system according to claim 11, wherein an outer peripheral surface of at least a distal end portion of the elongated tubular body of the catheter is coated with a lubricious coat.

15. The catheter system according to claim 11, wherein an effective length of the elongated tubular body of the catheter is less than 1000 mm.

16. A treatment method for causing at least part of a catheter system to pass through a lesion of a vein together with a guide wire, the treatment method comprising:
allowing passage through a lesion of a vein by the catheter system in which an elongated tubular body of a catheter is inserted into a guiding catheter, the catheter including the elongated tubular body having an inner diameter of not less than 1.4 mm and an outer diameter of not more than 2.8 mm, and a lesion passage catheter is inserted into the elongated tubular body.

17. The treatment method according to claim 16, wherein passage through a lesion of a vein is allowed by the catheter system in which the guiding catheter is inserted into an outer catheter, and an inner catheter is inserted into the lesion passage catheter.

18. The treatment method according to claim 16, wherein the outer diameter of the elongated tubular body is not more than 2.0 mm.

19. The treatment method according to claim 16, further comprising:
coating an outer peripheral surface of at least a distal end portion of the elongated tubular body with a lubricious coat.

20. The treatment method according to claim 16, wherein an effective length of the elongated tubular body is less than 1000 mm.
